(19) United States
(12) Patent Application Publication
(10) Pub. No.: US 2001/0002257 A1
(43) Pub. Date: May 31, 2001

(54) Composition comprising a lipoamino acid and the constituents of a tannin-rich plant extract, and cosmetic use thereof

(76) Inventor: Corinne Stolz, Paris (FR)

Correspondence Address: E. Joseph Gess, Esq., BURNS, DOANE, SWECKER & MATHIS, L.L.P., P.O. Box 1404, Alexandria, VA, 22313-1404, US

(21) Appl. No.: 09/754,209

(22) Filed: Jan. 5, 2001

Related U.S. Application Data
(63) Continuation of application No. 09/104,210, filed on Jun. 25, 1998.

(30) Foreign Application Priority Data
Jun. 25, 1997 (FR) 97-07943

(51) Int. Cl. A61K 31/195; A01N 37/12; A01N 37/44; A61K 35/78; A01N 65/00

(52) U.S. Cl. 424/401; 514/561; 424/70.1; 424/739; 424/729; 424/750; 424/780; 514/494; 424/773; 514/783

(57) ABSTRACT

Composition characterized in that it comprises, as active principle, at least one compound of formula (I):

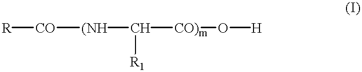

or its topically acceptable salts,

in which R represents the characterizing chain or a saturated or unsaturated, linear or branched fatty acid containing from 3 to 30 carbon atoms, R₁ represents a characterizing chain of an amino acid and m is between 1 and 5, and the constituents of at least one extract and/or of at least one tincture from tannin-rich plant starting materials (II).

The present invention relates to a novel cosmetic composition of compounds of lipoamino acid structure and having germicidal activity.

Most of the problems associated with skin, such as dandruff, comedones, cysts, blackheads or any other manifestations affecting the appearance of the human body, are associated with the presence of bacterial microorganisms which, in particular on account of their enzymes, induce a skin reaction such as, for example, an inflammation.

Among these skin problems, mention may be made of acne, which is a common skin complaint affecting, at the date of filing of the present patent application, about five million people in this country; the most common form is juvenile polymorphic acne which appears at puberty.

Acne is triggered in the pilosebaceous follicles by simultaneously inducing hyperkeratinization of the pilosebaceous ducts, hypersecretion of sebum and bacterial proliferation in the pilosebaceous follicles.

Hyperkeratinization of the pilosebaceous ducts leads to them becoming blocked, which in turn favours the implantation of a microorganism often found in this pathology:

Propionibacterium acnes.

The hypersecretion of sebum is a constant factor in the course of acne; it arises from increased sensitivity of the sebaceous gland to androgens, which leads to the increased secretion of sebum. On account of the hyperkeratinization of the pilosebaceous duct, the removal of sebum is impeded, or even prevented, and this leads to the formation of comedones and closed microcysts, The sebum thus accumulated in a follicle then becomes a site for bacterial proliferation. The sebum, which is rich in triglycerides, is then very rapidly degraded into free fatty acids (FFAS) by the lipases in the bacterial microorganisms. The free fatty acids thus formed become oxidized on contact with atmospheric oxygen, in particular into peroxides, which sustain or even aggravate the local inflammation.

Whether it is on healthy or acneic skin, the bacterial flora encountered on the skin surface or inside comedones is qualitatively the same: it consists of yeasts such as Pityrosporum ovale and Pityrosporum orbiculare, staphylococci such as Staphylococcus epidermis, Staphylococcus capitis or Staphylococcus hominis or propionibacteria such as Propionibacterium acnes.

Propionibacterium acnes produces lipases which are capable of hydrolysing the sebum triglycerides into free fatty acids. Free fatty acids are known to be comedogenic, i.e. they can give rise to follicular hyperkeratosis. Thus, colonization of the follicles by these bacteria is another source of comedogenic material which, thereafter, will give rise to the development of microcomedones. Propionibacterium acnes is also responsible for the accumulation of neutrophilic leukocytes and, indirectly, of mononuclear lymphocytes, thus leading to the development of an inflammation and the actuation of an immune response.

The staphylococci referred to above colonize both healthy skin and acneic lesions, in which they are found at a frequency comparable to that of Propionibacterium acnes; 70 to 75% of comedones are colonized, either with staphylococci or with propionibacteria, with on average 10⁴ to 10⁵ bacteria per comedone. The fact that comedones are open (blackheads) or closed (microcysts) does not appear to have a qualitative influence on the proliferation of these two types of bacteria. Staphylococcus epidermis secretes elastase, which is responsible for perifollicular elastolysis lesions.

The Pityrosporum yeasts, such as those mentioned above, are present in amounts of the same magnitude as the staphylococci and the propionibacteria; although aerobic, they can be encountered in the deep layers of follicles as well as in closed comedones.

The Applicant has thus sought to develop a composition which is effective against the damage caused by the microorganisms present on the skin, and in particular against the damage caused by the enzymes which sustain and develop the inflammatory process.

Compounds of lipoamino acid structure such as, for example, those described in the international patent applications published under the numbers WO 92/20647, WO 92/21318, WO 94/26694 and WO 94/27561 are, on account of their amphiphilic structure, biological vectors that are particularly advantageous as regulators of skin physiology and prove to be suitable for many different applications, in particular in cosmetics.

Now, the Applicant has found that compositions comprising, as active principles, a combination of lipoamino acids with certain natural plant extracts have both, on the one hand, germicidal and antimicrobial activity and, on the other hand, anti-inflammatory activity by inhibiting lipases, 5-alpha-reductase, which is responsible in particular for the production of sebum, and by inhibiting elastase. The Applicant has also found that this activity is the result of synergism between these two families of active principles.

The subject of the invention is a composition characterized in that it comprises, as active principle, at least one compound of formula (I):

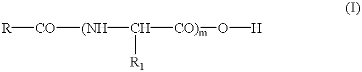

or its topically acceptable salts,

in which R represents the characterizing chain of a saturated or unsaturated, linear or branched fatty acid containing from 3 to 30 carbon atoms, R₁ represents a characterizing chain of an amino acid and m is between 1 and 5, and the constituents of at least one extract and/or of at least one tincture from tannin-rich plant starting materials (II).

The expression topically acceptable salt is understood to refer to any salt of the acid of formula (I) which is biologically acceptable to the skin and/or mucous membranes, i.e. any salt which can in particular adjust the pH of the composition to a value between 3 and 8 and preferably equal to about 5, i.e. a pH in the region of that of the skin.

The salts may be, in particular, alkali metal salts such as sodium, potassium or lithium salts, alkaline-earth metal salts such as calcium, magnesium or strontium salts; they can also be metal salts such as divalent zinc or manganese salts or alternatively trivalent iron, lanthanum, cerium or aluminium salts.

The compound of formula (I) present in the composition which is the subject of the present invention can be in free acid form or in partially or totally salified form.

The expression “characterizing chain” used in the context of the present application denotes the main non-functional chain of the fatty acid or of the amino acid considered.

Thus, for a fatty acid corresponding to the general formula R—COOH, the characterizing chain will be the chain represented by R.

The radical R in particular represents a radical containing from 5 to 22 carbon atoms, chosen from pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, uneicosyl, docosyl, heptadecenyl, eicosenyl, uneicosenyl, docosenyl, heptadecadienyl or decenyl radicals.

The subject of the invention is, more particularly, the composition as described above for which, in formula (I), the fragment R—CO contains from 7 to 22 carbon atoms and represents in particular one of the following radicals: hexanoyl, heptanoyl, octanoyl (capryloyl), decanoyl (caproyl), undecylenoyl, dodecanoyl (lauroyl), tetradecanoyl (myristyl), hexadecanoyl (palmitoyl), octadecanoyl (stearyl), eicosanoyl (arachidoyl), docosanoyl (behenoyl), octadecenoyl (oleyl), eicosenoyl (gadoloyl), docosenoyl (erucyl), octadecadienoyl (linolenoyl).

In a first preferred variant of the present invention, in formula (I), the fragment R—CO (I) contains from 7 to 12 carbon atoms.

For an amino acid represented by the general formula

H₂N—CHR₁—COOH,

the characterizing chain will be the chain represented by R₁.

R₁ represents in particular the characterizing chain of one of the amino acids chosen from glycine, alanine, serine, aspartic acid, glutamic acid, valine, threonine, arginine, lysine, proline, leucine, phenylalanine, isoleucine, histidine, tyrosine, tryptophan, asparagine, cysteine, cystine, methionine, hydroxyproline, hydroxylysine and ornithine.

The subject of the invention is, more particularly, the composition as described above for which, in formula (I), R₁ represents the characterizing chain of glycine, alanine, glutamic acid or aspartic acid.

The expression at least one compound of formula (I) indicates that the composition according to the invention can contain one or more of these compounds.

In the definition of the composition which is the subject of the present invention, the words extract and tincture are used in their respective meanings as established in the 1997 edition of the European Pharmaopoeia: extracts are concentrated preparations that are liquid, solid or of intermediate consistency, which are generally obtained from dried plant or animal starting materials. Tinctures are preparations generally obtained from dried plant or animal starting materials.

The expression extracts or tinctures from tannin-rich plant starting materials is understood to denote in particular extracts or tinctures of rathania, of tea, of cinnamon, of willow or of witch hazel. These extracts or tinctures are commercially available. Some of them are listed in the French and/or European Pharmacopoeias.

The subject of the invention is, more particularly, the composition as described above in which the extract or tincture of tannin-rich plant starting materials is an extract of cinnamon.

The compounds of formula (I) are generally obtained by acylation of compounds of formula (I′)

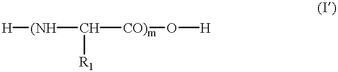

or of their salts, which are themselves obtained by total or partial hydrolysis of proteins of any origin. These proteins can be of animal origin, such as, for example, collagen, elastin, protein from fish flesh, fish gelatin, keratin or casein, of plant origin, such as, for example, those obtained from soybean, from sunflower, from oat, from wheat, from corn, from barley, from potato, from lupin, from horse bean, from sweet almond or from silk, or alternatively obtained from chorellae (unicellular algae), from rose algae or from yeasts.

This hydrolysis can be carried out, for example, by heating a protein placed in an acidic or alkaline medium to temperatures of between 60 and 130° C.

This hydrolysis can also be carried out enzymatically with a protease, optionally coupled with an alkaline or acidic post-hydrolysis.

When m is greater than 1, R₁ represents several of the characterizing chains of amino acids, depending on the protein hydrolysed and the degree of hydrolysis.

In a preferred variant of the present invention, when the composition comprises only one compound of formula (I), m is equal to 1 and when the composition comprises a mixture of compounds of formula (I), the average degree of condensation of the N-acylated amino acids in this mixture is less than 2.

The acylation reaction which allows the compounds of the abovementioned formula (I) to be obtained can be carried out chemically in alkaline medium (pH from 8 to 10) according to the Schotten-Baumann reaction or enzymatically, and a person skilled in the art can refer in particular to the reference work Surfactant Science Series, volume 7, Anionic Surfactants, part II, chapter 16, pages 581 to 617 (Marcel Dekker—1976).

In general, the embodiment currently preferred for the preparation of the lipoamino acid compounds of formula (I) comprises the following steps:

a) acylation in alkaline medium (pH 8 to 10) of an excess of amino acid mixture (extemporaneous mixture or mixture obtained by complete hydrolysis of a protein) with a fatty acid (or a mixture of fatty acids), in acid chloride or anhydride form.

The amino acid/acid chloride ratio is preferably from 1.05 to 1.30 equivalents.

The optimum acylation temperature is about 80° C. but varies, from one amino acid to another, between 60 and 110° C.

The duration of acylation depends on the equipment used (size, stirring); it is about 2 hours for an acylated mass of 500 kg and about 5 hours for an acylated mass of 5000 kg.

b) Breaking of the alkaline acylate by acidification in order to separate the water-soluble impurities by settling and to release the acidic organic acylate (optimum pH from 0.5 to 3 depending on the amino acids).

c) Purification by washing with water or with addition of electrolytes or of co-solvent in order to promote the separation by settling.

Besides the active principles, the composition according to the invention comprises inorganic or organic vehicles commonly used in the manufacture of compositions intended to be formulated as preparations for cosmetic and/or pharmaceutical use; mention may be made, for example, of water, propylene glycol, butylene glycol, hexylene glycol or glycerol.

The composition according to the invention, may also comprises one or more neutralizing agents commonly used in the manufacture of product for cosmetic; mention may be be made, for example, of Tris(trishydroxyaminomethane), triethylamine, lysine or methyl glycine.

In a preferred aspect of the present invention, the composition as described above comprises from 15% to 60%, and more particularly from 20% to 40%, by weight of at least one compound of formula (I) in which the fragment R—CO comprises from 7 to 12 carbon atoms, or its topically acceptable salts, and from 0.1% to 10% by weight, and more particularly from 0.5% to 5% by weight, of constituents of at least one extract and/or a tincture of tannin-rich plant starting materials.

According to the European Pharmacopoeia, the extracts can be in the form of fluid extracts, soft or hard extracts or dry extracts.

In the above definition of the composition according to the invention, the weight percentages of constituents of the extract or of the tincture correspond to the weight percentages of dry residue, the said dry residue being obtained by evaporation of the solvent and desiccation of the said extract or of the said tincture under operating conditions in which adverse changes of the constitutents are minimal.

The composition as defined above comprises in particular from 20% to 40% by weight of a compound of formula (I) in which R₁ represents a hydrogen atom, the fragment R—CO represents an octanoyl radical and m is equal to 1 and from 0.50% to 5% by weight of constituents of an extract of cinnamon.

As cinnamon, there is, for example, the cinnamon obtained from the Ceylonese cinnamon tree (Cinnamomum zeylanicum).

The composition according to the invention can also comprise from 0.1% to 10% by weight of magnesium potassium aspartate.

In a preferred aspect of the present invention, the composition as previously described comprises from 4% to 20% of lauroylglutamic acid, from 4% to 20% of lauroylaspartic acid, from 2% to 8% of lauroylglycine and from 2% to 8% of lauroylalanine, from 0.5% to 5% by weight of constituents of an extract of cinnamon and from 0.5% to 5% of magnesium potassium aspartate.

In another preferred aspect of the present invention, the composition as described above also comprises from 0.1% to 10% by weight of zinc gluconate.

The composition which is the subject of the present invention is prepared by methods known to those skilled in the art. In general, at least one extract and/or a tincture of tannin-rich plant starting materials is introduced, with stirring, into a composition comprising at least one compound of formula (I), this introduction being in an amount which is suitable to reach the desired concentration of constituents of the said extract and/or of the said tincture in the final solution. If necessary, or if desired, the said extract and/or the said tincture are prediluted before mixing.

The composition according to the invention is used in cosmetics. As shown by the following examples, the composition according to the invention is characterized, unexpectedly, by anti-elastase, anti-lipase, antibacterial and anti-5-α-reductase quadruple activity.

This makes it possible to use it, in particular, to soothe and/or protect sensitive skin, to moisturize dry skin, to slow down ageing of the skin and/or to treat acne-prone skin; in the latter indication, the composition according to the invention can be used as a complementary treatment to the medical treatment of acne.

The composition according to the invention is also used to disinfect the skin and mucous membranes. In this case, it can be a simple act of body hygiene or a complementary treatment to the medical treatment of an infection.

The composition according to the invention is also used to treat the scalp, in particular as an antidandruff active agent.

These uses also constitute in themselves a subject of the present invention.

Depending on the use, the composition as described above is used at various concentrations and in a formulation which is suitable for this use; such cosmetic compositions are usually in the form of aqueous solutions, dilute alcoholic solutions, or simple or multiple emulsions such as water-in-oil (W/O), oil-in-water (O/W) or water-in-oil-in-water (W/O/W) emulsions. As cosmetic formulations, mention may be made of creams, milks, lotions, wipes, shower gels, soaps, liquid soaps, “sondais”, intimate-hygiene products or shampoos.

Such formulations are known to those skilled in the art; their preparations are described, for example, in the patent applications published under the numbers WO 92/06778, WO 93/28204, WO 95/13863, WO 95/35089 or WO 96/22109.

The subject of the invention is thus also a cosmetic formulation which can be obtained by 1/10-fold to 1/20,000-fold dilution of the composition as described above, in one or more cosmetically acceptable excipients, and in particular a cosmetic formulation in the form of an oil-in-water emulsion having the appearance of a milk with a viscosity of less than 1 Pa.s, comprising a self-emulsifiable composition based on fatty alcohols as emulsifier.

As preferred self-emulsifiable composition, mention may be made of Montanov® 68 sold by the company SEPPIC.

The term dilution used hereinabove encompasses, in its widest accepted sense, all the steps which make it possible to go from the composition as defined above to the cosmetic formulation intended for sale.

In another preferred embodiment of the present invention, the cosmetic formulation is a lotion for treating acne-prone skin.

In another preferred embodiment of the present invention, the cosmetic formulation is a foaming formulation or an antidandruff shampoo.

The subject of the invention is particularly a cosmetic formulation comprising, as active principle, from 0.001% to 6% by weight of at least one compound of formula (I) and from 0.00005% to 1% by weight of constituents of at least one extract (II) and/or of at least one tincture of tannin-rich plant starting materials chosen from extracts of cinnamon, of rathania, of tea, of willow or of witch hazel, and, if so desired, up to 1% of zinc gluconate.

The subject of the invention is, most particularly, a cosmetic formulation comprising, as active principle, from 0.5% to 2% by weight of a compound of formula (I)₁

CH₃—(CH₂)₆—CO—NH—CH₂—COOH (I)₁

from 0.05% to 0.2% by weight of constituents of an extract of plant starting material chosen from extracts of cinnamon, of rathania, of tea, of willow or of witch hazel, and, if so desired, from 0.025% to 0.2% of zinc gluconate,

and a cosmetic formulation comprising, as active principle, from 0.001% to 0.005% by weight of at least one compound of formula (I)₁

CH₃—(CH₂)₆—CO—NH—CH₂—COOH (I)₁

from 0.0001% to 0.005% by weight of constituents of an extract of plant starting material chosen from extracts of cinnamon, of rathania, of tea, of willow or of witch hazel, and, if so desired, from 0.0001% to 0.0005% of zinc gluconate.

The examples which follow illustrate the invention without, however, limiting it.

### EXAMPLES:

### A) PREPARATION OF THE COMPOSITIONS ACCORDING TO THE INVENTION

1) Composition A

The following compounds are mixed together with stirring:

Lipacide® C8G sold by the company SEPPIC, the active principle of which is octanoylglycine

butylene glycol

glycerol

tris (tris(hydroxymethyl)aminomethane or tromethamine): quantity sufficient to obtain a pH of about 5.0 to 6.0;

Sepicide® HB (sold by the company SEPPIC)

water.

A liquid, stable, odourless and water-soluble solution with a pH of about 5.6 is thus obtained, to which an aqueous solution of a dry extract of cinnamon sold by the company Alban Muller International is added, with stirring; the mixture thus obtained is filtered through a membrane filter (about 3μ) in order to obtain composition A containing about:

25% by weight of octanoylglycine;

10% by weight of glycerol;

10% by weight of butylene glycol; and

3% by weight of constituents of extract of cinnamon.

2) Composition B

a) 96 kg of caustic potash (50%) are added to a mixture consisting of 27.4 kg of L-aspartic acid, 35.3 kg of L-glutamic acid, 7.9 kg of L-alanine, 7.9 kg of glycine and 112.5 kg of water, in order to obtain a pH of about 10.

A co-solvent to facilitate the subsequent acylation can also be added to this mixture.

The mixture thus obtained is brought to a temperature of 80° C. and 110 kg of lauroyl chloride are then run in with simultaneous stirring. The reaction lasts for 2 hours.

The “acylated mass” of 465 kg thus obtained comprises a mixture of lauroylamino acids, free amino acids and lauric acid, these compounds being in potassium salt form.

The mixture of compounds of lipoamino acid structure can be extracted from this acylated mass in very acidic medium (pH of about 1) and under hot conditions (90° C.), for example by carrying out the following procedure:

adjusting the pH to a value of 1 by adding an appropriate amount (about 125 kg) of 30% hydrochloric acid, at a temperature of about 80° C.;

stirring the mixture thus obtained for 30 minutes at 90° C. (temperature due to the exothermicity);

separating the phases by settling for 30 minutes (the compounds of lipoamino acid structure remaining in the organic phase);

removing the acidic waters, which are discarded after neutralization in order thus to eliminate the non-acylated free amino acids, the salt formed and the co-solvent;

washing the supernatant organic phase with salt water (10% NaCl);

removing the aqueous phase;

obtaining an organic phase containing more than 70% by weight of the following mixture of compounds of lipoamino acid structure, of formula (I):

| | |
|---|---|
| lauroylglutamic acid | 27% |
| lauroylaspartic acid | 30% |
| lauroylglycine acid | 6% |
| lauroylalanine acid | 8% |

along with reaction residues, in particular:

| | |
|---|---|
| lauric acid | 15% |
| water | 10% |
| sodium chloride | 1% |
| co-solvent (propylene glycol) | 3% |

b) The compounds below are added, with stirring, to 155 kg of the organic phase obtained in Example 1:

propylene glycol: 100 kg;

tris (tris(hydroxymethyl)aminomethane or tromethamine): quantity sufficient to obtain a pH of about 5.0 to 6.0;

Sepicide® HB: 2 kg:

water: 200 kg;

potassium magnesium aspartate: 5 kg.

The mixture thus obtained is filtered through a pocket filter (about 3μ).

A liquid, stable, odourless and water-soluble solution with a pH of about 5.7 is thus obtained, to which is added an aqueous solution of an extract of cinnamon in order to obtain composition B containing about:

25% by weight of the mixture of products of formula (I)

3% by weight of active material, of extract of cinnamon

1.25% by weight of magnesium potassium aspartate.

The following compositions were also prepared;

| | Products of | Extract of |
|---|---|---|
| Compositions | formula (I) | cinnamon |
| C | 25% lauroylglutamic acid | 3% |
| D | 25% lauroylglycine acid | 3% |
| E | 25% lauroylaspartic acid | 3% |
| F | 25% lauroylalanine acid | 3% |
| G | 25% undecylenoylglycine | 3% |
| H | 25% octanoylglycine | 2% |

### B) DEMONSTRATION OF THE ANTI-ELASTASE PROPERTIES OF THE COMPOSITIONS ACCORDING TO THE INVENTION.

a) Principle of the test:

It is known that human leukocytic elastase (HLE) is involved in a great many inflammatory pathologies. This enzyme is capable, in particular, of degrading many macromolecules such as fibrous elastin, certain types of collagen, proteoglycans and glycoproteins.

For this reason, human leukocytic elastase constitutes one of the links in the chain of reactions accompanying an inflammation phenomenon.

Blocking this enzyme by means of an anti-elastase effect thus makes it possible to prevent the degradation of the abovementioned molecules and thus to inhibit the inflammation process.

The anti-elastase properties of a given product can be demonstrated by means of an in vitro test, carried out with a spectrophotometer, using a support substance which can degrade as it becomes coloured, in contact with human leukocytic elastase.

Such a substance can be, for example, N-methoxysuccinyl-alanine-proline-valine-para-nitroanilide, a normally colourless substance which releases, by hydrolysis with human leukocytic elastase, a coloured product, para-nitroaniline, whose appearance kinetics can be monitored by spectrophotometry at 410 nm.

The reaction is carried out in a spectrophotometer thermostatically set at 25° C. and equipped with a sample changer. All of the kinetics are carried out a minimum of three times, the average and the standard deviation then being calculated for the three values obtained.

The presence of a molecule with anti-elastase activity is reflected by a limitation of the appearance of the coloured product and the anti-elastase effect can then be calculated relative to a control curve obtained in the absence or the said molecule.

A correlation thus exists between the percentage of inhibition of the appearance of the coloured product by the test compound and the percentage of inhibition of human leukocytic elastase.

The percentage of inhibition thus calculated is also representative of the calmant activity of the test compound.

Results obtained

Compositions A and H are diluted in water in order to obtain the compositions A₁, A₂, A₃, H₁, H₂ and H₃ having the following active principle concentrations:

| | octanoylglycine | extracts of cinnamon |
|---|---|---|
| A₁ | 0.625% | 0.0755% |
| A₂ | 0.002% | 0.00025% |
| A₃ | 0.0015% | 0.00018% |
| H₁ | 0.625% | 0.050% |
| H₂ | 0.002% | 0.00016% |
| H₃ | 0.0015% | 0.000125% |

The percentages of inhibition of elastase are as follows:

| | % inhibition |
|---|---|
| Composition A₁ | 100% |
| Composition A₂ | 70% |
| Composition A₂ | 54% |
| Composition H₁ | 100% |
| Composition H₂ | 55% |
| Composition H₃ | 43% |
| 0.625% octanoylglycine alone | 5% |
| extract of cinnamon at 0.00025% | 60% |
| extract of cinnamon at 0.00018% | 43% |
| extract of cinnamon at 0.00016% | 43% |
| extract of cinnamon at 0.000125% | 33% |
| extract of cinnamon at 0.2% | 100% |

These results show that at concentrations for which Lipacide® C8G has no inhibitory activity on elastase, and for which cinnamon has limited activity (from 33% to 60%), the combination of the two unexpectedly has higher activity.

### C) DEMONSTRATION OF THE ANTI-5α-REDUCTASE PROPERTIES OF THE COMPOSITIONS ACCORDING TO THE INVENTION.

a) Principle of the test

Enzymes of the 5-alpha-reductase type convert testosterone into 5-dehydrotestosterone (5-HT). The activity of 5-alpha-reductase is important on the skin, in particular in the perineal region. It is present in the sebaceous glands and the apocrine glands (sweat glands), in keratinocytes and fibroblasts.

Carbon-14-labelled testosterone is incubated in the presence of human skin microsomes, prepared according to the method described by Voigt et al. (1970) J. Biol. Chem., 245, 5594-5599, in the absence and in the presence of the test compound or in the presence of finasteride, used as reference inhibitory molecule.

After extraction and drying of the steroids, chromatography and quantification of the NADPH-dependent conversion of C-testosterone, the test results are expressed as a percentage of inhibition of the 5-alpha-reductase activity.

b) results

Using this test with a composition according to the invention containing Lipacide C8G and an extract of cinnamon, synergism was observed between these two products.

Using this test with a Lipacide C8G and zinc gluconate combination, synergism inherent in the combination of these two compounds was also observed for this activity.

For this activity, it is thus advantageous to combine the compounds of formula (I) with extracts of tannin-rich plant starting materials and/or zinc gluconate.

### D) DEMONSTRATION OF THE ANTI-LIPASE ACTIVITY OF THE COMPOSITIONS ACCORDING TO THE INVENTION.

a) Principle of the test

The test involves assaying the free fatty acids obtained by degradation of the triglycerides with lipase.

A known amount of triglycerides (“Lipase Substrate” sold by the company Sigma), a known amount of lipase (“Enzyme Control 2E” sold by the company Sigma) and the test composition are placed together in a tube. After an incubation period of three hours at 37° C., the enzymatic reaction is stopped and the free fatty acids formed are assayed by titration with 0.05 M sodium hydroxide. The results are expressed as a percentage of inhibition of the enzyme by the composition considered, by comparing it with a control test.

b) results

Composition A was diluted 1/15-fold in water in order to obtain composition A₄ having the following active principle concentrations:

octanoylglycine: 1.6%

extract of cinnamon: 0.19%.

Whereas the 1.6% octanoylglycine leads to a percentage of inhibition of 44% and the 0.19% extract of cinnamon leads to a percentage of inhibition of 16%, the composition A₄ leads to a percentage of inhibition of 71%, whereas the simple addition of the effects would give 60% inhibition.

This clearly demonstrates the synergism which exists between the two active principles.

It was observed that under the same operating conditions, a 0.13% solution of zinc gluconate leads to a percentage of inhibition of 40% and that the combination of octanoylglycine (1.6%) and zinc gluconate (0.13%) leads to a percentage of inhibition of 95%.

This test shows the advantage in using compositions according to the invention containing both compounds of formula (I), extracts of tannin-rich plant starting materials and/or zinc gluconate.

### E) DEMONSTRATION OF THE ANTI-MICROBIAL ACTIVITY ACCORDING TO THE INVENTION.

a) The minimum inhibitory concentration, at pH 5, of composition according to the invention on the growth of the strains below was determined:

Propionibacterium acnes

Staphylococcus aureus

Staphylococcus epidermis.

The following results are obtained:

| | | (II) | (I) + (II) |
|---|---|---|---|
| | (I) | extract of | combination |
| | octanoylglycine | cinnamon | of the |
| | alone | alone | products |
| P. acnes | 0.5% | 2% | 0.25% (I) + |
| | | | 0.5% (II) |
| Staph. | 0.25% | 2.5% | 0.10% (I) + |
| Aureus | | | 0.5% (II) |
| Staph. | 0.20% | 2% | 0.10% (I) + |
| Epidermis | | | 0.25% (II) |

These results reveal the synergism of the antimicrobial activity inherent in the combination of the products (I) and (II).

b) The minimum inhibitory concentration, at pH 5, on the growth of the same strains for an octanoylglycine+zinc gluconate combination was determined.

The following results are obtained:

| | | (III) | |
|---|---|---|---|
| | (I) | zinc | |
| | octanoylglycine | gluconate | combination |
| | alone | alone | (I) + (III) |
| P. acnes | 0.5% | 1% | 0.15% (I) + |
| | | | 0.25% (III) |
| Staph. | 0.25% | 0.5% | 0.005% (I) + |
| Aureus | | | 0.20% (III) |
| Staph. | 0.20% | 10.5% | 0.1% (I) + |
| Epidermis | | | 0.20% (III) |

These results reveal the synergism of the antimicrobial activity inherent in the combination of the products (I)+(III).

These tests show the advantage of combining, in the same formulation, a product of formula (I) and an extract of tannin-rich plant starting materials and/or of zinc gluconate.

### F) PREPARATION OF COSMETIC FORMULATIONS

### Example 1: Care formulation for greasy and acne-prone skin.

| | FORMULATION | | |
|---|---|---|---|
| A | Montanov ® 202 | | |
| | (Arachidyl behenyl alcohols & | | 3.00% |
| | Arachidylglucoside - SEPPIC) | | |
| B | Water | q.s. | 100% |

| | | |
|---|---|---|
| | Composition A | 4.00% |
| C | Dimethicone | 4.00% |
| | Isohexadecane | 8.00% |
| D | Sepigel ® 305 | 2.00% |
| | (polyacrylamide/C13-14 isoparaffin/ | |
| | Laureth-7-SEPPIC) | |
| E | Sepicide ® HB | 0.50% |
| | (phenoxyethanol/methylparaben/ethylparaben/ | |
| | propylparaben/ | |
| | butylparaben - SEPPIC) | |
| | Fragrance | 0.20% |

Procedure: Heat phase B to 85° C. Add Montanov® 202 (flakes) and stir until completely melted. Add phase C without preheating. Homogenize with the emulsifier, Start cooling very slowly. At 70° C., introduce the Sepigel. At 60° C., stop the emulsifier, place under vacuum and continue cooling with slow stirring. At about 30° C., introduce the constituents of phase E.

### Example 2: Anti-sheen cream-gel for greasy or combination skin.

| | FORMULATION: | | |
|---|---|---|---|
| A | Sepigel ® 305 | | 3.50% |
| | (Polyacrylamide/C13-14 isoparaffin/Laureth | | |
| | 7-SEPPIC) | | |
| | Pelemol ® 89 | | 5.00% |
| | (Octyl isononanoate - Phoenix) | | |
| | Micropearl ® M305 | | 1.00% |
| | (crosslinked polymethyl methacrylate- | | |
| | SEPPIC) | | |
| | Water | q.s. | 100% |

| | | |
|---|---|---|
| B | Composition A | 4.00% |
| | Sepicide ® HB | 0.30% |
| | (Phenoxyethanol/methylparaben/ | |
| | ethylparaben/propylparaben/ | |
| | butylparaben-SEPPIC) | |
| | Sepicide ® CI | 0.20% |
| | (Imidazolidinylurea-SEPPIC) | |
| | Fragrance | 0.10% |
| C | Capigel ® 98 | 1.00% |
| | (Acrylates copolymer-SEPPIC) | |
| | Water | 10.00% |

Procedure: Introduce the Micropearl® M305 into the Pelemol®, add the Sepigel® 305 and then raise the gel with water. Next, add composition A, followed by all the other constituents of phase B. Next, add the Capigel® 98 prediluted in water. Adjust the pH to about 7.

### Example 3: Purifying cleansing formulation for greasy, acne-prone skin

| FORMULATION | | |
|---|---|---|
| Water | q.s. | 100% |
| Metolose ® 65SH4000 (hydroxypropyl | | 1.00% |
| methylcellulose-SEPPIC) | | |
| Base Nacrante [pearlescent base] 9578 | | 10.00% |
| (sodium laureth sulphate/cocamide DEA/ | | |
| glycol stearate-SEPPIC) | | |
| Proteol ® Oat (sodium lauroyl oat | | [lacuna] |
| amino acids-SEPPIC) | | |
| Composition A | | 2.00% |
| Capigel ® 98 | | 2.00% |
| (acrylates copolymer-SEPPIC) | | |
| Water | | 10.00% |
| Fragrance | | 0.20% |
| Sepicide ® HB (phenoxyethanol/ | | 0.20% |
| methylparaben/ethylparaben/propylparaben/ | | |
| butylparaben-SEPPIC) | | |
| Trometamol | q.s | pH 7.2 |

Procedure: Disperse the Metolose in hot water at about 70-75° C. and leave to cool. At about 30° C., add the pearlescent base, the Proteol Oat and then composition A, with stirring. Next, add the acrylate copolymer predispersed in the 10% of water. Next, introduce the fragrance and the preserving agent. Neutralize the formulation to the desired pH.

### Example 4: Moisturizing cream for greasy skin

| FORMULATION | | |
|---|---|---|
| Montanov ® 68 | | 5% |
| Cetylstearyl octanoate | | 8% |
| Composition A | | 2 to 4% |
| Water | q.s. | 100% |
| Compound of Example 1: | | 0.6% |
| Micropearl ® M100 | | 3.0% |
| Mucopolysaccharides | | 5% |
| Sepicide ® HB | | 0.8% |
| Fragrance | | 0.3% |

### Example 5: Alcohol-free soothing after-shave balm

| FORMULATION | | |
|---|---|---|
| Composition A | | 2 to 4% |
| Lanol ® 99 | | 2% |
| Sweet almond oil | | 0.5% |
| Water | q.s. | 100% |
| Compound of Example 1: | | 3% |
| Sepicide ® HB | | 0.3% |
| Sepicide ® CI | | 0.2% |
| Fragrance | | 0.4% |

(Lanol ® 99 is isononyl isononanoate sold by the company SEPPIC)

### Example 6: Cream with AHAs for sensitive skin

| FORMULATION | | |
|---|---|---|
| Composition A | | 2% |
| Magnesium potassium aspartate | | 0.002% to 0.5% |
| Lanol ® 99 | | 2% |
| Montanov ® 68 | | 5.0% |
| Water | q.s. | 100% |
| Compound of Example 1 | | 1.50% |
| Triethylamine | | 0.9% |
| Sepicide ® MB | | 0.3% |
| Sepicide ® CI | | 0.2% |
| Fragrance | | 0.4% |

1. Composition characterized in that it comprises, as active principle, at least one compound of formula (I): 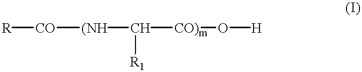
or its topically acceptable salts,
in which R represents the characterizing chain of a saturated or unsaturated, linear or branched fatty acid containing from 3 to 30 carbon atoms, R₁ represents a characterizing chain of an amino acid and m is between 1 and 5, and the constituents of at least one extract and/or of at least one tincture from tannin-rich plant starting materials (II):

2. Composition as defined in claim 1, for which, in formula (I), the fragment R—CO contains from 7 to 22 carbon atoms and represents in particular one of the following radicals: hexanoyl, heptanoyl, octanoyl (capryloyl), decanoyl (caproyl), undecylenoyl, dodecanoyl (lauroyl), tetradecanoyl (myristyl), hexadecanoyl (palmitoyl), octadecanoyl (stearyl), eicosanoyl (arachidoyl), docosanoyl (behenoyl), octadecenoyl (oleyl), eicosenoyl (gadoloyl), docosenoyl (erucyl), octadecadienoyl (linolenoyl).

3. Composition as defined in either of claims 1 and 2, for which, in formula (I), the fragment R—CO (I) contains from 7 to 12 carbon atoms.

4. Composition defined in any one of claims 1 to 3, for which, in formula (I), R₁ represents the characterizing chain of glycine, alanine, glutamic acid or aspartic acid.

5. Composition as defined in any one of claims 1 to 4, in which the extract and/or the tincture of tannin-rich plant starting materials is chosen from the extracts of rathania, of tea, of cinnamon, of willow and of witch hazel.

6. Composition as defined in any one of claims 1 to 5, in which the extract and/or the tincture of tannin-rich plant starting materials is an extract of cinnamon.

7. Composition as defined in any one of claims 1 to 6, characterized in that when the composition comprises only one compound of formula (I), m is equal to 1 and when the composition comprises a mixture of compounds of formula (I), the average degree of condensation of the N-acylated amino acids in this mixture is less than 2.

8. Composition as defined in claim 3, comprising from 15% to 60% by weight of at least one compound of formula (I) or one of its topically acceptable salts, and from 0.1% to 10% by weight of constituents of at least one extract and/or a tincture of tannin-rich plant starting materials.

9. Composition as defined in claim 8, comprising from 20% to 40% by weight of at least one compound of formula (I) in which R₁ represents a hydrogen atom, the fragment R—CO represents an octanoyl radical and m is equal to 1 and from 0.50% to 5% by weight of constituents of an extract of cinnamon.

10. Composition as defined in one of claims 1 to 9, also comprising from 0.1% to 10% by weight of magnesium potassium aspartate.

11. Composition as defined in claim 10, comprising from 4% to 20% of lauroylglutamic acid, from 4% to 20% of lauroylaspartic acid, from 2% to 8% of lauroylglycine and from 2% to 8% of lauroylalanine, from 0.5% to 5% by weight of constituents of an extract of cinnamon and from 0.5% to 5% of magnesium potassium aspartate.

12. Composition as defined in one of claims 1 to 11, characterized in that it also contains zinc gluconate.

13. Use of the composition as defined in one of claims 1 to 12, in cosmetics.

14. Use according to claim 13, for soothing and/or protecting sensitive skin, for moisturizing dry skin, and/or for slowing down ageing of the skin, and/or for treating the scalp.

15. Use according to claim 13, for disinfecting the skin and mucous membranes.

16. Use according to claim 13, for treating acne-prone skin.

17. Cosmetic formulation which can be obtained by 1/10-fold to 1/20,000-fold dilution of the composition as defined by any one of claims 1 to 12, in one or more cosmetically acceptable excipients.

18. Formulation as defined in claim 17, in the form of an oil-in-water emulsion having the appearance of a milk with a viscosity of less than 1 Pa.s, comprising a self-emulsifiable composition based on fatty alcohols as emulsifier.

19. Formulation as defined in claim 17, in the form of a lotion for treating acne-prone skin.

20. Formulation as defined in claim 17, in the form of a foaming formulation or an antidandruff shampoo.

21. Cosmetic formulation comprising, as active principle, from 0.001 to 6% by weight of at least one compound of formula (I) and from 0.00005% to 1% by weight of constituents of an extract (II) of tannin-rich plant starting materials.

22. Cosmetic formulation as defined in claim 19, comprising, as active principle, from 0.5% to 2% by weight or a compound of formula (I)₁
CH₃—(CH₂)₆—CO—NH—CH₂—COOH (I)₁
from 0.05% to 0.2% by weight of constituents of an extract of plant starting materials chosen from extracts of cinnamon, of rathania, of tea, of willow or of witch hazel.

23. Cosmetic formulation as defined in claim 22, also comprising 0.025% to 0.2% of zinc gluconate.

24. Cosmetic formulation as defined in claim 21, comprising, as active principle, from 0.001% to 0.005% by weight of a compound of formula (I)₁
CH₃—(CH₂)₆—CO—NH—CH₂—COOH (I)₁
from 0.0001% to 0.005% by weight of constituents of an extract of plant starting materials chosen from extracts of cinnamon, of rathania, of tea, of willow or of witch hazel.

25. Cosmetic formulation as defined in claim 24, comprising from 0.0001% to 0.0005% of zinc gluconate.
